(19) United States
(12) Patent Application Publication
(10) Pub. No.: US 2007/0249720 A1
(43) Pub. Date: Oct. 25, 2007

(54) Thiourea Derivative-Containing Pharmaceutical Composition Having Improved Solubility and Bioavailability

(76) Inventors: Jae Hyun Kim, Chungcheongnam-do (KR); Joo Hyun Moh, Kyunggi-do (KR); Young Ho Park, Seoul (KR); Jung Ju Kim, Gyeonggi-do (KR); Young Chul Shim, Gyeonggi-do (KR); Joon Ho Bae, Seoul (KR)

Correspondence Address: HESLIN ROTHENBERG FARLEY & MESITI PC, 5 COLUMBIA CIRCLE, ALBANY, NY, 12203, US

(21) Appl. No.: 10/576,759

(22) PCT Filed: Oct. 22, 2004
(86) PCT No.: PCT/KR04/02702

§ 371 Date: Mar. 2, 2007

(30) Foreign Application Priority Data
Oct. 23, 2003 (KR) 10-2003-0074274

(51) Int. Cl. A61K 31/17; A61P 1/04; A61P 11/06; A61P 13/10; A61P 17/00; A61P 25/00; A61P 25/06; A61P 9/00; C07C 335/42

(52) U.S. Cl. 514/586; 564/1.5

(57) ABSTRACT

The present invention relates to a pharmaceutical composition comprising a thiourea derivative or its pharmaceutically acceptable salt, a cyclodextrin or its derivative; and a pharmaceutical formulation comprising same.

### TECHNICAL FIELD

The present invention relates to a pharmaceutical composition comprising a thiourea derivative or its pharmaceutically acceptable salt, a cyclodextrin or its derivative; and a pharmaceutical formulation comprising same.

### BACKGROUND ART

Capsaicin (8-methyl-N-vanillyl-6-nonenamide) is a main pungent component of hot pepper. Hot pepper has been used for a long time, not only as a spice but also as a traditional medicine for the treatment of gastric disorders and, when applied topically, for the relief of pain and inflammation (Szallasi and Blumberg, Pharm, Rev., 51, pp 159-212(1999)). Capsaicin has a wide spectrum of physiological activities: it exhibits strong irritant effects on the cardiovascular and respiratory systems and also induces pain and irritancy upon topical application. However, after such induction of pain, capsaicin induces desensitization both to capsaicin itself and also to other noxious stimuli, thereby producing analgesic effect. Based on this property, capsaicin and its analogues such as olvanil, nuvanil, DA-5018, SDZ-249482, and resiniferatoxin are used as an analgesic agent, or a therapeutic agent for incontinentia urinae or skin disorder (Wriggleworth and Walpole, Drugs of the Future, 23, pp 531-538(1998)).

Mechanical, thermal and chemical noxious stimuli are mainly transmitted by the primary afferent nerve fibers such as non-medullated nerve fiber(C-fiber) and medullated nerve fiber(A-fiber), and capsaicin and its analogs(“vanilloid”) act on such nerve fibers. Capsaicin acts on a receptor present on the nerve fibers to induce a sharp stimulus by causing a potent inflow of mono- and di-valent cations such as calcium and sodium ions, and then blocks the nerve function, thereby resulting in a strong analgesic effect (Wood et al., J. Neurosci, 8, pp 3208-3220(1988)). Vanilloid receptor (VR1) was cloned very recently, thereby its presence was confirmed (Caterina et al., Nature, 389, pp 783-784 (1997)). It has been reported that the receptor of vanilloid on the nerve fibers, i.e., vanilloid receptor (VR1), transmits not only stimuli by capsaicin or vanilloid but also various noxious stimuli such as proton and thermal stimuli (Tominaga et al., Neuron, 21, pp 531-543 (1998)). These facts suggest that vanilloid receptor functions as an integrative modulator against various noxious stimuli and carries out a critical role in the transmissions of pain and noxious stimuli. Recently, a knockout mouse lacking the vanilloid receptor gene was prepared (Caterina et al., 2000, Science, 288, pp 306-313; Davis et al., 2000, Nature, 405, pp 183-187), which exhibited a significantly reduced reactivity to thermal stimuli and thermal hyperalgesia as compared to normal mice. This result reconfirms the importance of the receptor in the transmission of noxious stimuli.

As mentioned above, capsaicin-responsive sensory nerve cells and vanilloid receptors existing thereon are distributed over the whole body, and play the basic function of transmitting pain and noxious stimuli. Moreover, they together further act as a crucial factor in the expression of neurogenic inflammation, and, accordingly, are closely related with the cause of a disease such as neuropathies, nerve injury, stroke, asthma, chronic obstructive pulmonary diseases, urinary bladder hypersensitiveness, irritable bowel syndrome, inflammatory bowel disease, fervescence, skin disorder and inflammatory diseases. Their connection with a neuropathic disease was also suggested (WO 99/00125). Recently, attention has been paid to the role of the afferent sensory nerve responding to capsaicin upon gastrointestinal injury. It has also been proposed that the afferent nerve might improve gastric microcirculation and exhibit a protective activity against gastric injury by releasing peripheral neuropeptide such as CGRP (calcitonin gene-related peptide), while inducing gastric injury by stimulating sympathetic nerve system (Ren et al., Dig. Dis. Sci., 45, pp 830-836(2000)). Accordingly, vanilloid receptor modulators are expected to be a potent medicine for preventing or treating said various diseases by modulating the activity of the multi-functional vanilloid receptor.

In WO 02/16318, the present inventors clearly demonstrated through animal tests the analgesic, anti-inflammatory and anti-ulcerous effects of numerous vanilloid receptor antagonists including thiourea derivatives, thereby suggesting the availability of a vanilloid receptor antagonist as an analgesic, anti-inflammatory and anti-ulcerous agent. However, such thiourea derivatives are hardly water-soluble and, accordingly, it is difficult to make a liquid formulation, e.g., an injectable solution, containing same in a pharmacologically effective amount. Further, a solid formulation containing same has many problems when used clinically, since it exhibits limited bioavailability and significant individual variation in the plasma drug concentration. Accordingly, there still exists a need to develop a means to increase the solubility and bioavailability of the thiourea derivatives.

For this, many researchers in our laboratory have conducted various studies to improve dissolution or bioavailability of thiourea derivatives with low water-solubility by using various carriers and formulation methods, yet every trial was ended in fail because of high lipophilicity of the thiourea derivatives. For example, in the case of an attempt to raise dissolution rate via reducing particle size of drug by comminuting enabled the formation of solid preparation, but formation of liquid preparation was impossible. The percent dissolution was not significantly improved. Solid dispersion showed almost semisolid appearance due to property of drug itself and the percent dissolution was also low.

Cyclodextrins are cyclic compounds having d-glucopyranose units linked with α-(1→4)glycosidic bonds. The outer surface of a cyclodextrin is hydrophilic due to the presence of hydroxyl groups thereon, while its interior is hydrophobic. Accordingly, a lipophilic substance having a molecular structure fittable to the interior of the cyclodextrin (“guest molecule”) may be included in the cyclodextrin to form an inclusion complex. Generally used cyclodextrins are α-, β-, and γ-cyclodextrins having 6, 7 and 8 glucopyranose units, respectively, among which β-cyclodextrins are preferred due to its inclusion potency and low cost. Compounds forming inclusion complexes with cyclodextrins are reported in Journal of Parenteral Science & Technology, 43, pp 231-240 (1989) and Stella and Rajewski, Pharmaceutical Research, 14, pp 556-567 (1997).

Recently, various cyclodextrin derivatives having high solubilities were developed, examples of which include alkyl-cyclodextrin, hydroxyalkyl-cyclodextrin, carboxyethyl-cyclodextrin, sulfoalkylether-cyclodextrin, etc. As a hydroxyalkyl, preferred is that having C₁₋₆ alkyl group, e.g. hydroxymethyl, hydroxyethyl, hydroxypropyl and hydroxybutyl etc., and hydroxypropyl is particularly preferred. Among the various cyclodextrin derivatives, 2-hydroxypropyl-β-cyclodextrin is most suitable for use in an injection and oral formulations, because it is highly soluble in water and non-toxic. Various cyclodextrin derivatives are reported in Rajewski and Stella, Journal of Pharmaceutical Science 85(11), pp 1142-1169 (1996).

With regard to the inclusion complexes using cyclodextrins, U.S. Pat. No. 4,727,064 discloses a method for improving pharmaceutical properties. For example, low water solubility of a lipophilic drug may be improved by dissolving a cyclodextrin derivative in an aqueous median and adding the drug to the resulting solution to form a drug/cyclodextrin complex. U.S. Pat. No. 4,596,795 discloses that the administration by the sublingual or buccal route of a sex hormone in the form of its inclusion complex with a cyclodextrin derivative results in effective transfer of the hormone into the systemic circulation, followed by only gradual degradation. Further, U.S. Pat. No. 4,371,673 discloses cyclodextrin complexes of retinoid-polymers, and complexes of retinoids with ether type derivatives of cyclodextrins.

### DISCLOSURE OF INVENTION

Accordingly, it is an object of the present invention to provide a pharmaceutical composition having an improved solubility and bioavailability of a thiourea derivative having an excellent antagonistic activity against vanilloid receptor 1 (VR1).

It is another object of the present invention to provide a pharmaceutical formulation containing said composition, which has an improved homogeneity, safety and bioavailability.

In accordance with one aspect of the present invention, there is provided a pharmaceutical composition comprising: a thiourea derivative of formula (I) or its pharmaceutically acceptable salt, a cyclodextrin or its derivative, and, optionally, a pharmaceutically acceptable additive:

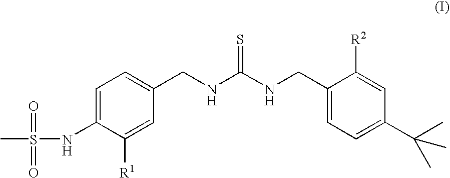

wherein,

R¹ is hydrogen, fluoro, chloro, methoxycarbonyl, carboxyl or hydroxyaminocarbonyl, and

R² is hydrogen, methoxy, ethoxy, propoxy, butoxy, isopropoxy, isobutoxy, neopentoxy, methoxymethoxy or benzyloxy.

In accordance with another aspect of the present invention, there is provided a pharmaceutical formulation comprising said pharmaceutical composition for preventing or treating a disease selected from the group consisting of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, asthma, chronic obstructive pulmonary disease, irritation of skin, eye or mucous membrane, fervescence, stomach-duodenal ulcer, and inflammatory diseases.

In another aspect the invention relates to an inclusion complex comprising a thiourea derivative of formula (I)

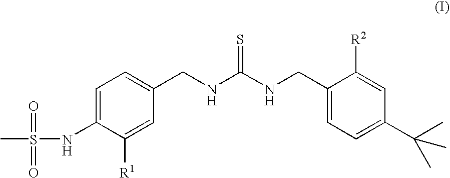

wherein,

R¹ is hydrogen, fluoro, chloro, methoxycarbonyl, carboxyl or hydroxyaminocarbonyl, and

R² is hydrogen, methoxy, ethoxy, propoxy, butoxy, isopropoxy, isobutoxy, neopentoxy, methoxymethoxy or benzyloxy;

or a pharmaceutically acceptable salt thereof, and a cyclodextrin or cyclodextrin derivative.

The invention further relates to the use of an inclusion complex of a thiourea derivative of formula I and a cyclodextrin or its derivative for preparing a medicament for treating a disease associated with the pathological stimulation and/or increased expression of vanilloid receptors.

The invention further relates to the method of treating a mammal including man suffering from the pathological stimulation of VR1 receptors comprising administering to said mammal a pharmaceutical composition comprising a thiourea derivative of formula (I) or its pharmaceutically acceptable salt, a cyclodextrin or its derivative, and, optionally, a pharmaceutically acceptable additive.

The invention further relates to the use of pharmaceutical composition comprising a thiourea derivative of formula (I) or its pharmaceutically acceptable salt, a cyclodextrin or its derivative, and, optionally, a pharmaceutically acceptable additive, for treating a disease associated with the pathological stimulation and/or increased expression of vanilloid receptors.

The thiourea derivative of formula (I) are disclosed in WO 02/16318 and may be prepared in accordance with a process as disclosed therein.

Preferred thiourea derivatives for use in the present invention are

1-(4-t-butylbenzyl)-3-(3-fluoro-4-methanesulfonylaminobenzyl)thiourea,

1-(4-t-butylbenzyl)-3-(3-chloro-4-methanesulfonylaminobenzyl)thiourea,

1-(4-t-butylbenzyl)-3-(3-methoxycarbonyl-4-methanesulfonyl-aminobenzyl)thiourea,

1-(4-t-butylbenzyl)-3-(4-methanesulfonylaminobenzyl)thiourea,

1-(4-t-butyl-2-isobutoxybenzyl)-3-(4-methanesulfonylaminobenzyl)thiourea, and pharmaceutically acceptable salts thereof.

Among them, particularly preferred is 1-(4-t-butylbenzyl)-3-(3-fluoro-4-methanesulfonylaminobenzyl)thiourea.

The inventive pharmaceutical composition comprises a cyclodextrin or its derivative as a solubility and bioavailability-improving carrier for the thiourea derivative of formula (I) or its pharmaceutically acceptable salt. The inventive pharmaceutical composition may comprise the cyclodextrin or its derivative in an amount ranging from 1 to 50 parts by weight, preferably 1 to 20 parts by weight per 1 part of the thiourea derivative or its pharmaceutically acceptable salt.

The cyclodextrin may be of an anhydrous or hydrated form. Further, it may be either amorphous or crystalline, or α-, β- or γ-type.

Preferred examples of a cyclodextrin derivative which may be used in the present invention include α-, β- or γ-cyclodextrin derivatives wherein at least one hydroxyl group of the cyclodextrin is substituted. Suitable substituents are for example alkyl or substituted alkyl groups such as methyl, ethyl, hydroxyethyl, hydroxypropyl, hydroxybutyl, carboxymethyl, or carboxyethyl (an ether derivative); a saccharide such as maltosyl, glucosyl, or maltotriosyl (a saccharide derivative); or a sulfoalkyl group (a sulfoalkyl ether derivative).

Preferred cyclodextrin derivatives may be 2,6-dimethyl-β-cyclodextrin, 2-hydroxyethyl-β-cyclodextrin, 2-hydroxypropyl-β-cyclodextrin, 2-hydroxyethyl-γ-cyclodextrin, 2-hydroxypropyl-γ-cyclodextrin, (2-carboxymethoxy)propyl-β-cyclodextrin or sulfobutylether-7-β-cyclodextrin, and particularly preferred is 2-hydroxypropyl-β-cyclodextrin. Further, an amorphous cyclodextrin derivative may be preferably employed in the present invention.

The inventive composition may further comprise a pharmaceutically acceptable additive known in the art, e.g., an electrolytic or non-electrolytic diluent, pH controller, osmotic controller, buffer, flavor, binder, thickener, lubricant and preservative, and a mixture thereof.

When the inventive pharmaceutical composition is exposed to water or gastro-intestinal juices, the water-soluble carrier in the form of minute solid particles is released to the aqueous phase and, simultaneously, the components of the inclusion complex and/or solid dispersion are released as minute particles, thereby increasing the surface area of a drug particle. As the drug particles become smaller and the carrier dissolves completely in a very short time, the solubilization of the drug by the carrier is achieved within the diffusion layer, the minute environment surrounding drug particles at the early stage of dissolution. Therefore, it is understood that the above-mentioned factors work collectively to increase the solubility and initial dissolution rate of the drug.

Further, when exposed to water or aqueous body fluids, the inclusion complex of the thiourea derivative/cyclodextrin or its derivative may form a supersaturated solution of the drug, through a process in which the insoluble thiourea derivative is included in the hydrophobic cavity of the highly water-soluble cyclodextrin or its derivative while the latter dissolves in water.

The inventive pharmaceutical composition may be prepared by a method comprising the steps of (a) uniformly homogenizing a cyclodextrin or its derivative in an aqueous solution such as water or a buffer or in an organic solvent such as an alcohol, e.g., ethanol, (b) reacting the resulting cyclodextrin solution with a thiourea derivative while stirring, and optionally, (c) drying the resulting reaction product, e.g., by lyophilization, vacuum-drying, spray-drying, or fluid bed drying, to obtain a solid powder.

Representative examples of the organic solvent include chloroform, dichloromethane, methanol, ethanol, propanol, isopropanol, methylethylketone, acetone, diethylether, dimethylether, tetrahydrofuran, cyclohexane, and ethyl acetate. Preferred is ethanol.

When an aqueous solution or a small amount of ethanol is used in the homogenizing step of the cyclodextrin or its derivative, the liquid phase reaction product obtained in step b) may be used, only after filtering, in the preparation of an injectable solution or an internal liquid formulation.

The solid powder obtained in step c) may be sieved or pulverized to have appropriately-sized particles, and then used in the preparation of a solid formulation. This solid product has advantages in that it has an improved solubility causing reduction of individual variation in the plasma drug concentration and that it is in the form of a fluidizable powder suitable for the preparation of a solid formulation.

As the thiourea derivative of formula (I) has a lower solubility in an aqueous solution than in an organic solvent, if an aqueous solution is used as a medium in the step a), the resulting pharmaceutical composition comprises mainly an inclusion complex of the thiourea derivative and the cyclodextrin. On the other hand, when an organic solvent is used in the step a), the resulting pharmaceutical composition comprises mainly a solid dispersion of the thiourea derivative and cyclodextrin.

The inventive composition which may be in the form of an inclusion complex and/or solid dispersion of the thiourea derivative and cyclodextrin or its derivative exhibits an excellent solubility and a high dissolution rate of the thiourea derivative in water or a gastrointestinal liquid, which leads to increased bioavailability.

The inventive pharmaceutical composition may be combined with a pharmaceutically acceptable excipient to provide a pharmaceutical formulation, which can be administered orally or non-orally, e.g., by an intravenous, subcutaneous, intramuscular, transdermal, transocular, transnasal, intravaginal or intrarectal injection. Preferably, the inventive composition is administered orally. The pharmaceutical formulation may further comprise known other active ingredients, in addition to the inventive pharmaceutical composition.

The pharmaceutical formulation for an oral administration may be a solid type such as a tablet, pill, powder, granule, pellet or capsule, or a liquid type such as a solution, suspension or syrup. The oral formulation may be rapidly releasable or sustained releasable.

For example, the solid type oral formulation may contain conventional pharmaceutically acceptable excipients such as a binder (e.g., pre-gelatinized corn starch, polyvinylpyrrolidone or hydroxypropylmethylcellulose), filler for directly tableting (e.g., spray-dried lactose, microcrystalline cellulose or calcium hydrogen phosphate), lubricant (e.g., magnesium stearate, talc, silica or sodium stearyl fumarate) or surfactant (e.g., sodium lauryl sulfate or polysorbate).

The tablet formulation may be coated using a conventionally known method. For example, a saccharide, beeswax or a combination thereof, or a water-soluble polymer such as polyvinylpyrrolidone, polyvinylalcohol or hydroxypropyl cellulose may be used as a coating material which disintegrates in the mouth or stomach; and alternatively, a gastric liquid-resistant material may be used as a coating material so that the active ingredients are absorbed at the intestine or the colon.

Liquid for oral administration can have a form such as solutions, syrups or suspensions (for example, composition coated with gastric fluid-resistant coating material and composition dispersed as particles in water or suspension such as syrup), or can be provided as a dry composition which is mixed with water or other suitable excipient prior to use.

The coated tablet, granule or pellet may comprise a coated film layer and a nucleus. The film layer may be made of at least one film forming material selected from cellulose acetate, ethyl cellulose, cellulose acetate phthalate, hydroxypropylmethyl cellulose, hydroxypropylmethyl cellulose phthalate, wax, Eudragits, hydroxypropyl cellulose acetate succinate, etc., or at least one channel forming material selected from polyethyleneglycol, sorbitol, sucrose, an organic acid, etc., or a combination thereof.

The capsule formulation may be obtained by filling powders, granules or solutions into a capsule made of, e.g., gelatin.

Preferred solid type oral formulation may be an osmotic pump tablet, multilayer tablet, coated tablet, coated pallet, recombined powder, capsule, and coated granule.

The liquid type formulation for an oral administration such as a solution, syrup or suspension may be prepared in a conventional manner using an emulsifier (e.g., lecithin or acacia), non-aqueous solvent (e.g., almond oil, fatty ester, ethanol or fractionated vegetable oil), and preservative (e.g., methyl- or propyl-p-hydroxybenzoate, benzyl alcohol, or sorbic acid). The liquid formulation may be prepared by mixing a dried solid type formulation with a suitable aqueous or non-aqueous carrier, and it may further comprise an additional additive such as a pH controller, flavor, coloring agent or sweetening agent. Representative examples of the pH controller are acids including organic acids such as tartaric acid, citric acid, fumaric acid, maleic acid, malic acid, succinic acid, oxalic acid, benzoic acid, malonic acid, mandelic acid and ascorbic acid; and inorganic acids such as phosphoric acid, and bases such as sodium hydroxide and sodium carbonate.

The inventive pharmaceutical formulation for intravenous, subcutaneous, or intramuscular administration may be in the form of an injectable solution in which active ingredients are dissolved in a sterilized aqueous or non-aqueous solvent. Representative example of the aqueous solvent include physiological saline, and representative examples of the non-aqueous solvent are propylene glycol, polyethylene glycol, a vegetable oil such as olive oil, ethyl oleate, iodinated poppy oil and fatty acid ester. These formulations may further contain an additional additive such as an isotonic solution, preservative, wetting agent, emulsifier, dispersant or stabilizer, and they may be sterilized by filtering, mixing with an antibacterial agent or irradiating. These formulations may be prepared in the form of a solid formulation combined with a sterilized pyrogen-free substance so that they can be dissolved in a suitable solvent such as a sterilized distilled water or a physiological saline before use.

The inventive pharmaceutical formulation for transdermal administration may be in the form of an ointment, cream, lotion, liquid, gel, paste, patch, and aerosol, and it may be prepared in a conventional manner.

Further, the inventive pharmaceutical formulation for transocular administration may be preferably in the form of a liquid having a higher transparency than a suspension type formulation. It can be prepared in a solid formulation form, which can be dissolved in a suitable solvent before use. The transocular formulation may further comprise additional adjuvants such as a buffering agent, tonicity adjustion agent, thickener, suspending agent, solubilizer, pH controller, or a chelating agent. Representative examples of the buffering agent include a phosphate, boric acid, sodium borate, and an organic acid (e.g., acetic acid and citric acid) or its salt. Representative examples of the buffering agent include boric acid, an alkali metal salt (e.g., sodium chloride and potassium chloride), and glycerol. Representative examples of the thickener include hydroxypropylcellulose and its salts. Representative examples of suspending agent are a surfactant (e.g., polysorbate) and a water-soluble polymer (e.g., carboxymethyl cellulose sodium salt, hydroxypropyl methyl cellulose, methyl cellulose and polyvinyl alcohol). Representative examples of the solubilizer include a non-ionic surfactant, e.g., polyoxyethylene-hydrogenated castor oil, polyoxyethylene sorbitan monooleate, polyoxyethylene stearate, triglyceride, polyethylene glycol. Representative examples of the pH controller include an alkali compound (e.g., sodium hydroxide, sodium hydrogen phosphate, and sodium borate), and an acidic compound (e.g., hydrochloric, boric, phosphoric, or acetic acid). Suitable examples of the chelating agent are sodium ethylenediaminetetraacetate, sodium citrate, and condensed sodium phosphate.

The inventive pharmaceutical formulation for transnasal administration may be in the form of a solution or powder. In case of the solution form, it is preferably more transparent than an suspension type formulation, and it may be prepared in a powder or tablet formulation form capable of dissolving in a suitable solvent before use. Representative examples of such a solvent include water, saline, a phosphate buffer, and an acetate buffer. The solution type transnasal formulation may further comprise an additive such as a surfactant, an anti-oxidant, a stabilizer, a preservative and a thickener commonly known in the art. The powder type formulation may preferably comprise an absorptive base, representative examples of which include a water soluble base such as a polyacrylate salt (e.g., sodium polyacrylate, potassium polyacrylate, and ammonium polyacrylate), a lower alkyl ether of cellulose (e.g., methyl cellulose, hydroxyethyl cellulose, hydroxypropyl cellulose, sodium carboxymethyl cellulose), polyethyleneglycol, polyvinylpyrrolidone, amylose and pullulan; a water-insoluble base such as a cellulose derivative (e.g., crystalline cellulose, α-cellulose, crosslinked sodium carboxymethylcellulose), a dextrin derivative (e.g., hydroxypropyl dextrin, carboxymethyl dextrin, crosslinked dextrin, amylose, amylopectin, pectin), a protein (e.g., gelatin, casein, sodium casein), a gun (e.g., Arabic gum, tragacanth gun, glycomannan), polyvinyl pyrrolidone, a crosslinked polyacrylic acid or its salt, a crosslinked polyvinyl alcohol; and a mixture thereof. The powdery formulation may further comprise an additive such as an anti-oxidant, a colorant, a preservative and a storage stabilizer commonly known in the art. The solution or powder type pharmaceutical formulation for transnasal administration may be preferably administered using a spraying tool.

The composition of the present invention may be formulated into a liquid or semisolid intravaginal or intrarectal formulation, e.g., a suppository or supplementary enema comprising conventional suppository bases such as cocoa butters and glycerides.

The inventive composition may be administered to a target site as an inclusion complex and/or a solid dispersion by itself or as a powder or a liquid composition containing the inclusion complex and/or the solid dispersion in combination with appropriate biocompatible excipients, by using an apparatus for oral or transnasal administration, e.g., a spray, a nebulizer and an atomizer. The inventive composition may be also administered by suspending in propellant for aerosol, such as freon.

The pharmaceutical composition of the present invention or the inventive inclusion complex can be effectively used for preventing or treating diseases associated with the regulation of the vanilloid receptor. These disease can be caused by the increased expression or stimulation of a vanilloid receptor, e.g. of VR1, or these diseases may itself cause an abnormal stimulation, expression or otherwise pathological regulation of a vanilloid receptor, e.g. the VR1. Such diseases include, but are not limited to, pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, respiratory disorder such as asthma or chronic obstructive pulmonary diseases, irritation of skin, eye or mucous membrane, fervescence, stomach-duodenal ulcer, and inflammatory diseases. The pharmaceutical composition of the present invention or the inventive inclusion complex can be especially effectively used for preventing or treating pain.

The present invention also relates to methods of treating mammals including human patients suffering from the above mentioned diseases by administering to said mammals including human patients a pharmaceutical composition according to the present invention in a therapeutically effective amount.

### BRIEF DESCRIPTION OF THE DRAWINGS

The above and other objects and features of the present invention will become apparent from the following description of the invention, when taken in conjunction with the accompanying drawings, which respectively show:

FIG. 1: a graph comparing the percent dissolution (%) of 1-(4-t-butylbenzyl)-3-(3-fluoro-4-methanesulfonylaminobenzyl)thiourea raw powder (◯) with that of Formulation Example 2 (●); and

FIG. 2: a graph showing plasma concentration-time curves measured after the administration of 1-(4-t-butylbenzyl)-3-(3-fluoro-4-methanesulfonylaminobenzyl)thiourea suspension (◯) and Formulation Example 3 (●) to rats, respectively.

### MODE FOR THE INVENTION

The following Examples and Experimental examples are intended to further illustrate the present invention without limiting its scope.

Further, percentages given below for solid in solid mixture, liquid in liquid, and solid in liquid are on a wt/wt, vol/vol and wt/vol basis, respectively, and all the reactions were carried out at room temperature, unless specifically indicated otherwise.

### EXPERIMENTAL EXAMPLE 1

0.4 g of 1-(4-t-butylbenzyl)-3-(3-fluoro-4-methanesulfonylaminobenzyl)thiourea (Compound 1) was added to each 10 ml of 0, 1.5, 3.5, 7.0, 14.0 and 28.0 w/v % aqueous solutions of 2-hydroxypropyl-β-cyclodextrin. The resulting mixture was stirred for 72 hours, filtered through a 0.2 micrometer filter paper. The concentration of Compound 1 in the filtrate was determined by high performance liquid chromatography (HPLC) and the solubility of Compound 1 depending on the concentration of 2-hydroxypropyl-β-cyclodextrin is presented in Table 1.

TABLE 1

| Conc. of 2-hydroxypropyl-β-cyclodextrin (w/v %) | Solubility (mg/ml) |
|---|---|
| 0 | 0.01 |
| 1.5 | 0.40 |
| 3.5 | 1.04 |
| 7.0 | 4.18 |
| 14.0 | 12.23 |
| 28.0 | 28.99 |

The result in Table 1 shows that the solubility of Compound 1 becomes higher with the concentration of 2-hydroxypropyl-β-cyclodextrin.

### EXPERIMENTAL EXAMPLE 2

0.4 g of 1-(4-t-butylbenzyl)-3-(3-fluoro-4-methanesulfonylaminobenzyl)thiourea (Compound 1) was added to each 10 ml of 0, 1.5, 3.5, 7.0, 14.0 and 28.0 w/v % 2-hydroxypropyl-β-cyclodextrin solution in glycine buffer (pH 10.5). The resulting mixture was stirred for 72 hours, filtered through a 0.2 micrometer filter paper.

The concentration of Compound 1 in the filtrate was determined by high performance liquid chromatography (HPLC) and the solubility of Compound 1 depending on the concentration of 2-hylroxypropyl-β-cyclodextrin is presented in Table 2.

TABLE 2

| Conc. of 2-hydroxypropyl-β-cyclodextrin (w/v %) | Solubility (mg/ml) |
|---|---|
| 0 | 0.05 |
| 1.5 | 2.70 |
| 3.5 | 4.80 |
| 7.0 | 9.62 |
| 14.0 | 18.01 |
| 28.0 | 32.47 |

The result in Table 2 shows that the solubility of Compound 1 becomes higher with the concentration of 2-hydroxypropyl-β-cyclodextrin.

### EXAMPLES 1-3

14, 20 or 28 g of 2-hydroxypropyl-β-cyclodextrin was put to a volumetric flask, deionized water was added thereto up to 100 ml, and the mixture was stirred. 2 g of 1-(4-t-butylbenzyl)-3-(3-fluoro-4-methanesulfonylaminobenzyl)thiourea (Compound 1) was added thereto and the mixture was stirred until became transparent. The solution was filtered through 0.2 micrometer filter paper, and the filtrate was lyophilized to obtain a white solid, which was then passed through a #40 sieve.

TABLE 3

| | Ratio of Compound 1:2-hydroxypropyl-β-cyclodextrin |
|---|---|
| Example 1 | 2 g:14 g |
| Example 2 | 2 g:20 g |
| Example 3 | 2 g:28 g |

### EXPERIMENTAL EXAMPLE 3

5 g of 1-(4-t-butylbenzyl)-3-(3-fluoro-4-methanesulfonylaminobenzyl)thiourea (Compound 1) was added to 100 ml of 95% ethanol solution containing 0, 1.0, 2.0, 5.0, 10.0 or 20.0 w/v % of 2-hylroxypropyl-β-cyclodextrin, and the mixture was stirred for 72 hours and then filtered through a 0.2 micrometer filter paper. The concentration of Compound 1 in the filtrate was determined by high performance liquid chromatography (HPLC) and the solubility of Compound 1 depending on the concentration of 2-hydroxypropyl-β-cyclodextrin is presented in Table 4.

TABLE 4

| Conc. of 2-hydroxypropyl-β-cyclodextrin (w/v %) | Solubility (mg/ml) |
|---|---|
| 0 | 19.72 |
| 1.0 | 21.22 |
| 2.0 | 22.65 |
| 5.0 | 27.22 |
| 10.0 | 34.15 |
| 20.0 | 43.47 |

The result in Table 4 shows that the solubility of Compound 1 becomes higher with the concentration of 2-hydroxypropyl-β-cyclodextrin.

### EXAMPLES 4-6

15, 20 or 30 g of 2-hydroxypropyl-β-cyclodextrin was added to a volumetric flask, 95% ethanol was added thereto to a total volume of 100 ml, and the mixture was stirred. 4.5 g of thiourea derivative, 1-(4-t-butylbenzyl)-3-(3-fluoro-4-methanesulfonylaminobenzyl)thiourea (Compound 1) was added thereto and the mixture was shaken until Compound 1 was completely dissolved. The resulting solution was vacuum dried to remove the solvent, to obtain a white solid. The solid was passed through a #40 sieve.

TABLE 5

| | Ratio of Compound 1:2-hydroxypropyl-β-cyclodextrin |
|---|---|
| Example 4 | 4.5 g:15 g |
| Example 5 | 4.5 g:20 g |
| Example 6 | 4.5 g:30 g |

### FORMULATION EXAMPLES

The composition of the present invention can be prepared into various pharmaceutical formulations, alone or in combination with appropriate pharmaceutical excipients, according to any one of the conventional methods as exemplified below.

### Formulation Example 1

### Preparation of a Capsule

| | mg/capsule |
|---|---|
| 1-(4-t-butylbenzyl)-3-(3-fluoro-4- | 20 |
| methanesulfonylaminobenzyl)thiourea | |
| 2-hydroxypropyl-β-cyclodextrin | 280 |
| Magnesium stearate | 1 |

The white powder prepared in Example 3 was mixed thoroughly with magnesium stearate in a mixer according to the above composition and filled in a #0 capsule.

### Formulation Example 2

### Preparation of a Tablet

| | mg/tablet |
|---|---|
| 1-(4-t-butylbenzyl)-3-(3-fluoro-4- | 90 |
| methanesulfonylaminobenzyl)thiourea | |
| 2-hydroxypropyl-β-cyclodextrin | 400 |
| Magnesium stearate | 1 |

The white powder prepared in Example 5 was mixed thoroughly with magnesium stearate in a mixer according to the above composition, and subjected to a conventional tabletting process to obtain a tablet.

### Formulation Example 3

### Preparation of a Liquid Formulation

| | g/liquid formulation |
|---|---|
| 1-(4-t-butylbenzyl)-3-(3-fluoro-4- | 2 |
| methanesulfonylaminobenzyl)thiourea | |
| 2-hydroxypropyl-β-cyclodextrin | 28 |
| Deionized water | q.s. to a total volume of 100 ml |

2-hydroxypropyl-β-cyclodextrin was dissolved in deionized water while stirring, and then 1-(4-t-butylbenzyl)-3-(3-fluoro-4-methanesulfonylaminobenzyl)thiourea was added thereto and dissolved while stirring. The resulting solution was filtered through a sterilized 0.45 micrometer filter, and filled and sealed in a vial to obtain a liquid preparation.

### Formulation Example 4

### Preparation of an Injection Formulation

| | g/injection formulation |
|---|---|
| 1-(4-t-butylbenzyl)-3-(3-fluoro-4- | 2 |
| methanesulfonylaminobenzyl)thiourea | |
| 2-hydroxypropyl-β-cyclodextrin | 28 |
| Deionized water | q.s. to a total volume of 100 ml |

2-hydroxypropyl-β-cyclodextrin was dissolved in deionized water while stirring, and then 1-(4-t-butylbenzyl)-3-(3-fluoro-4-methanesulfonylaminobenzyl)thiourea was added thereto and dissolved while stirring. The resulting solution was filtered through a sterilized 0.2 micrometer filter, and then filled, lyophilized and sealed in a vial to obtain an injection preparation.

### Formulation Example 5

### Preparation of a Transdermal Gel Formulation

| | g/gel formulation |
|---|---|
| 1-(4-t-butylbenzyl)-3-(3-fluoro-4- | 2 |
| methanesulfonylaminobenzyl)thiourea | |
| 2-hydroxypropyl-β-cyclodextrin | 28 |
| Poloxamer | 20 |
| Hydroxypropylmethylcellulose | 0.5 |
| Deionized water | q.s. to a total volume of 100 ml |

The white powder prepared in Example 3 was mixed thoroughly with other ingredients to obtain a transdermal gel formulation.

### Experimental Example 4

### Comparative Dissolution Test

1-(4-t-butylbenzyl)-3-(3-fluoro-4-methanesulfonylaminobenzyl)thiourea (Compound 1) and the tablet prepared in Formulation Example 2 were subjected to test in accordance with dissolution test method II (Paddle method) described in Korean pharmacopoeia using water as a release solvent.

3 ml samples were taken at a given time interval under the condition of 37° C. and 50 rpm, and filtered through a 0.45 micrometer filter. The concentration of Compound 1 in each sample was determined by HPLC.

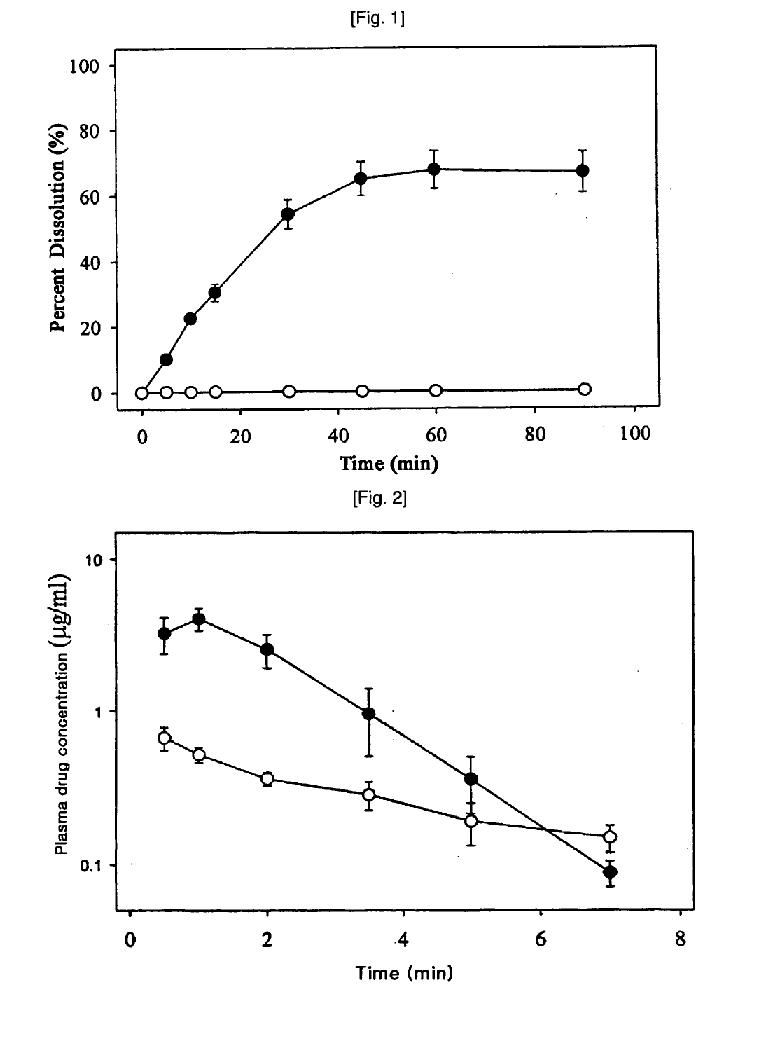

The time-dependent changes in the released amount of 1-(4-t-butylbenzyl)-3-(3-fluoro-4-methanesulfonylaminobenzyl)thiourea are shown in FIG. 1(●: the tablet of Formulation Example 2 and ◯: Compound 1). As shown in FIG. 1, the percent dissolution of drug from the inventive formulation increased significantly, while that from Compound 1 itself was insoluble in water.

### Experimental Example 5

### Test for Pharmacokinetics

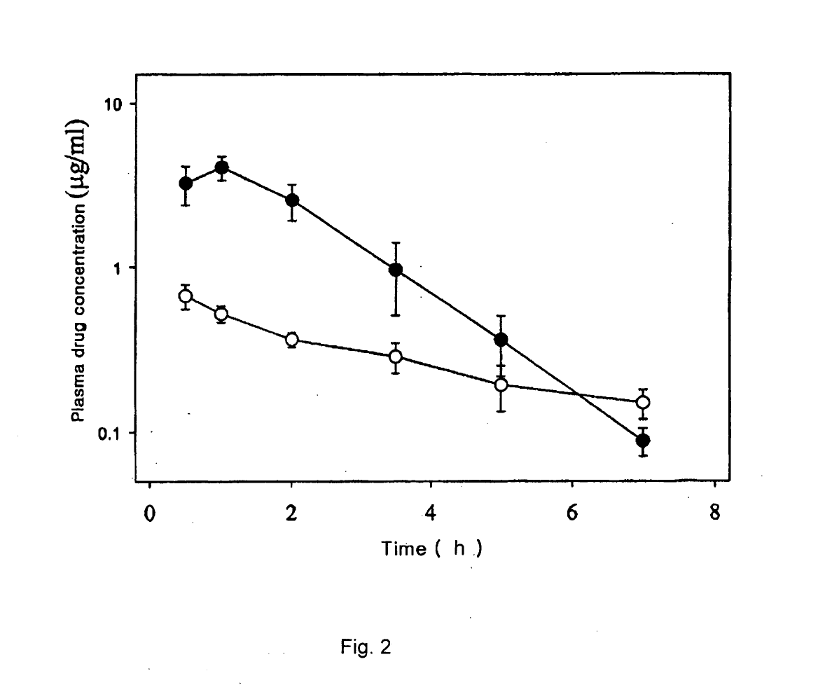

1-(4-t-Butylbenzyl)-3-(3-fluoro-4-methanesulfonylaminobenzyl)thiourea (Compound 1) and the liquid formulation prepared in Formulation Example 3 were each orally administered to rats, and the changes in the plasma drug concentration were monitored according to a set time schedule. Each experimental animal group consisted of four male rats. After the rats were anesthetized with ether and operated for inserting a PE-50 polyethylene tube into the femoral artery, the suspension of Compound 1 in 0.5% sodium carboxymethylcellulose solution and the liquid formulation of Formulation Example 3 were each orally administered to the rats. At 15, 30, 60, 120, 210, 300 and 480 minutes after the administration, 150 micro liter blood samples were taken from the rats. Each blood sample was centrifuged to separate plasma, which was then subjected to HPLC to determine the concentration of Compound 1 in plasma. The results are shown in Table 6 and FIG. 2.

TABLE 6
Comparison of pharmacokinetic parameters between Compound
1 and the liquid formulation of Formulation Example 3

| | | AUC*¹ | | | |
|---|---|---|---|---|---|
| | | (micro- | Cₘₐₓ*² | | |
| | | gram · | (micro- | Tₘₐₓ*³ | BA*⁴ |
| Test sample (Dose) | N | h/ml) | gram/ml) | (hr) | (%) |
| Liquid formulation of | 4 | 10.25 | 4.26 | 0.75 | 61.01 |
| Formulation Example 3 | | | | | |
| (10 mg/kg) | | | | | |
| Suspension of Compound 1 | 4 | 2.79 | 0.67 | 0.50 | 16.54 |
| (10 mg/kg) | | | | | |

*¹AUC: Area under the plasma concentration vs. time curve till 24 hours.
*²Cₘₐₓ: Maximum plasma concentration.
*³Tₘₐₓ: Time at the maximum plasma concentration.
*⁴BA: Bioavailability.

As can be seen the above results, the inventive formulation (Formulation Example 3) showed a significant difference in the time-dependent plasma concentration as compared with Compound 1 alone, and its bioavailability was also about 4-folds higher than Compound 1 due to its improved solubility and dissolution rate.

While the invention has been described with respect to the above specific embodiments, it should be recognized that various modifications and changes may be made to the invention by those skilled in the art which also fall within the scope of the invention as defined by the appended claims.

1. A pharmaceutical composition comprising: a thiourea derivative of formula (I) 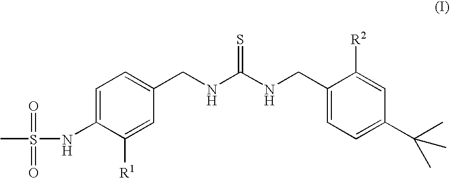
or a pharmaceutically acceptable salt thereof; and a cyclodextrin or its derivative,
wherein,
R¹ is hydrogen, fluoro, chloro, methoxycarbonyl, carboxyl or hydroxyaminocarbonyl, and
R² is hydrogen, methoxy, ethoxy, propoxy, butoxy, isopropoxy, isobutoxy, neopentoxy, methoxymethoxy or benzyloxy.

2. The pharmaceutical composition of claim 1, wherein the thiourea derivative is selected from the group consisting of:
1-(4-t-butylbenzyl)-3-(3-fluoro-4-methanesulfonylaminobenzyl)thiourea,
1-(4-t-butylbenzyl)-3-(3-chloro-4-methanesulfonylaminobenzyl)thiourea,
1-(4-t-butylbenzyl)-3-(3-methoxycarbonyl-4-methanesulfonyl-aminobenzyl)thiourea,
1-(4-t-butylbenzyl)-3-(4-methanesulfonylaminobenzyl)thiourea, and
1-(4-t-butyl-2-isobutoxybenzyl)-3-(4-methanesulfonylaminobenzyl)thiourea.

3. The pharmaceutical composition of claim 1, wherein the thiourea derivative is 1-(4-t-butylbenzyl)-3-(3-fluoro-4-methanesulfonylaminobenzyl)thiourea.

4. The pharmaceutical composition of claim 1 wherein cyclodextrin or its derivative is present in an amount ranging from 1 to 20 parts by weight per 1 part of the thiourea derivative or the pharmaceutically acceptable salt thereof.

5. The pharmaceutical composition of claim 1, wherein the cyclodextrin is of α-, β- or γ-type.

6. The pharmaceutical composition of claim 1, wherein the cyclodextrin derivative is selected from the group consisting of 2,6-dimethyl-β-cyclodextrin, 2-hydroxyethyl-β-cyclodextrin, 2-hydroxypropyl-β-cyclodextrin, 2-hydroxyethyl-γ-cyclodextrin, 2-hydroxypropyl-γ-cyclodextrin, (2-carboxymethoxy)propyl-β-cyclodextrin, and sulfobutylether-7-β-cyclodextrin.

7. The pharmaceutical composition of claim 1, wherein the cyclodextrin derivative is 2-hydroxypropyl-β-cyclodextrin.

8. The pharmaceutical composition of claim 1, wherein the composition further comprises a pharmaceutically acceptable additive.

9. The pharmaceutical composition of claim 8, wherein the pharmaceutically acceptable additive is selected from the group consisting of diluents, pH controllers, osmotic controller, buffers, flavors, binders, thickeners, lubricants, preservatives, and a combination thereof.

10. The pharmaceutical composition of claim 1, wherein the composition is in the form of a solution containing an inclusion complex prepared by dissolving the thiourea derivative or the pharmaceutically acceptable salt thereof and cyclodextrin or its derivative in water or a buffer.

11. The pharmaceutical composition of claim 1, which comprises a solid inclusion complex prepared by dissolving the thiourea derivative or the pharmaceutically acceptable salt thereof and the cyclodextrin or its derivative in water or a buffer, and subjecting the resulting solution to lyophilization, spray drying, vacuum drying or fluid bed drying to remove water.

12. The pharmaceutical composition of claim 1, which comprises a solid inclusion complex and/or a solid dispersion prepared by dissolving the thiourea derivative or the pharmaceutically acceptable salt thereof and the cyclodextrin or its derivative in an organic solvent, and subjecting the resulting solution to lyophilization, spray drying, vacuum drying or fluid bed drying to remove the organic solvent.

13. The pharmaceutical composition of claim 12, wherein the organic solvent is ethanol.

14. A pharmaceutical formulation comprising the pharmaceutical composition of claim 1 wherein the thiourea derivative of formula (I) is present in an amount being effective for preventing or treating a disease selected from the group consisting of pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, asthma, chronic obstructive pulmonary disease, irritation of skin, eye or mucous membrane, fervescence, stomach-duodenal ulcer, and inflammatory diseases.

15. The pharmaceutical formulation of claim 14, which is an oral formulation selected from the group consisting of a tablet, pill, powder, granule, solution, suspension, syrup and capsule.

16. The pharmaceutical formulation of claim 14, which is an injectable solution for intravenous, subcutaneous or intramuscular injection.

17. The pharmaceutical formulation of claim 14, which is a transdermal formulation selected from the group consisting of ointment, cream, lotion, solution, gel, paste, patch and aerosol.

18. The pharmaceutical formulation of claim 14, which is a liquid transocular formulation.

19. The pharmaceutical formulation of claim 14, which is a liquid or powder-type transnasal formulation.

20. The pharmaceutical formulation of claim 14, which is a liquid or semi-solid intravaginal or intrarectal formulation.

21. Inclusion complex comprising a thiourea derivative of formula (I) 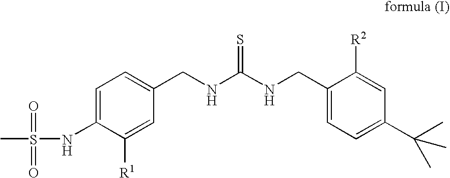
or a pharmaceutically acceptable salt thereof, and a cyclodextrin or its derivative,
wherein,
R¹ is hydrogen, fluoro, chloro, methoxycarbonyl, carboxyl or hydroxyaminocarbonyl, and
R² is hydrogen, methoxy, ethoxy, propoxy, butoxy, isopropoxy, isobutoxy, neopentoxy, methoxymethoxy or benzyloxy.

22. The inclusion complex of claim 21, wherein the thiourea derivative is selected from the group consisting of:
1-(4-t-butylbenzyl)-3-(3-fluoro-4-methanesulfonylaminobenzyl)thiourea,
1-(4-t-butylbenzyl)-3-(3-chloro-4-methanesulfonylaminobenzyl)thiourea,
1-(4-t-butylbenzyl)-3-(3-methoxycarbonyl-4-methanesulfonyl-aminobenzyl)thiourea,
1-(4-t-butylbenzyl)-3-(4-methanesulfonylaminobenzyl)thiourea, and
1-(4-t-butyl-2-isobutoxybenzyl)-3-(4-methanesulfonylaminobenzyl)thiourea.

23. The inclusion complex of claim 21, wherein the thiourea derivative is 1-(4-t-butylbenzyl)-3-(3-fluoro-4-methanesulfonylaminobenzyl)thiourea.

24. The inclusion complex of claim 21, which comprises the cyclodextrin or its derivative in an amount ranging from 1 to 20 parts by weight per 1 part of the thiourea derivative or the pharmaceutically acceptable salt thereof.

25. The inclusion complex of claim 21, wherein the cyclodextrin is of α-, β- or γ-type.

26. The inclusion complex of claim 21, wherein the cyclodextrin derivative is selected from the group consisting of 2,6-dimethyl-β-cyclodextrin, 2-hydroxyethyl-β-cyclodextrin, 2-hydroxypropyl-β-cyclodextrin, 2-hydroxyethyl-γ-cyclodextrin, 2-hydroxypropyl-γ-cyclodextrin, (2-carboxymethoxy)propyl-β-cyclodextrin, and sulfobutylether-7-β-cyclodextrin.

27. The inclusion complex of claim 21, wherein the cyclodextrin derivative is 2-hydroxypropyl-β-cyclodextrin.

28-30. (canceled)

31. Method of treating a mammal including man suffering from the pathological stimulation of VR1 receptors comprising administering to said mammal a therapeutically effective amount of the pharmaceutical composition according to claim 1.

32. Method according to claim 31, wherein the pathological stimulation of VR1 receptors is associated with at least one of the diseases selected from pain, acute pain, chronic pain, neuropathic pain, post-operative pain, migraine, arthralgia, neuropathies, nerve injury, diabetic neuropathy, neurodegeneration, neurotic skin disorder, stroke, urinary bladder hypersensitiveness, irritable bowel syndrome, asthma, chronic obstructive pulmonary disease, irritation of skin, eye or mucous membrane, fervescence, stomach-duodenal ulcer, and inflammatory diseases.

33-35. (canceled)
